(19) United States
(12) Patent Application Publication
(10) Pub. No.: US 2022/0318422 A1
(43) Pub. Date: Oct. 6, 2022

(54) METHODS, SYSTEMS, AND COMPUTER PROGRAM PRODUCTS FOR PROCESSING HEALTH CARE INFORMATION ACCESS REQUESTS BASED ON HIERARCHICAL ACCESS RULES

(71) Applicant: Change Healthcare Holdings LLC, Nashville, TN (US)

(72) Inventors: German Scipioni, San Jose, CA (US); David Juitt, Arlington, MA (US); Anthony David Guido, Corona, CA (US); Aubriana Menendez, San Francisco, CA (US); Alex Sheu, San Jose, CA (US); Spencer Cross, Oakland, CA (US); Allan Dip, Austin, TX (US)

(21) Appl. No.: 17/218,953

(22) Filed: Mar. 31, 2021

(51) Int. Cl. G06F 21/62; G16H 10/60

(52) U.S. Cl. CPC G06F 21/6245; G16H 10/60

(57) ABSTRACT

A method includes performing operations as follows on a processor: defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority; receiving information associated with health care services provided to a patient; receiving a request to access a portion of the information from a requesting source; and determining whether to grant the request based on the portion of the information, the requesting source, and the first and second health care information access rules.

### FIELD

The present inventive concepts relate generally to health care systems and services and, more particularly, to controlling access to patient health care information/data.

### BACKGROUND

The Centers for Medicare & Medicaid Services (CMS) recently mandated new rules regarding health information technology interoperability and a patient's right of access to his or her health information/data. The mandate affects the entire healthcare industry, but it may particularly affect the payor market. Payors may be expected to make a patient's health care information/data available to them electronically through a variety of electronic channels, including mobile applications, by allowing for secure access to data through interoperable application protocol interfaces (APIs). While these rules are expected to provide significant benefits to patients by increasing their ability to review and access their health care information/data, payors have the burden to develop systems including APIs to facilitate patient access while still complying with privacy laws and other laws, rules, and/or regulations that govern the handling of patients' health care information/data. Payors must also stay current with these laws, rules, and/or regulations for many different jurisdictions including, for example, federal, state and local governmental jurisdictions.

### SUMMARY

According to some embodiments of the inventive concept, a method comprises performing operations as follows on a processor: defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority; receiving information associated with health care services provided to a patient; receiving a request to access a portion of the information from a requesting source; and determining whether to grant the request based on the portion of the information, the requesting source, and the first and second health care information access rules.

In other embodiments, the first and second health care information access rules are based on health care information categories and relationship status roles between the requesting source and the patient.

In still other embodiments, the health care information categories comprise a claim information category, an encounter information category, a clinical information category, pharmacy information category, a formulary information category, and a wearable information category.

In still other embodiments, the claim information category comprises claim information associated with a current payor for the patient and a former payor for the patient.

In still other embodiments, the clinical information category comprises a plurality of clinical conditions.

In still other embodiments, each of the plurality of clinical conditions is associated with a plurality of clinical sub-conditions identifiable by a plurality of clinical codes, respectively.

In still other embodiments, each of the second health care information access rules has a hierarchy rating associated therewith, the hierarchy rating specifying a precedence for resolving conflicts between ones of the second health care information access rules.

In still other embodiments, the first governmental administrative authority is a federal government administrative authority and the second governmental administrative authority is a state government administrative authority.

In some embodiments of the inventive concept, a system comprises a processor; and a memory coupled to the processor and comprising computer readable program code embodied in the memory that is executable by the processor to perform operations comprising: defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority; receiving information associated with health care services provided to a patient; receiving a request to access a portion of the information from a requesting source; and determining whether to grant the request based on the portion of the information, the requesting source, and the first and second health care information access rules.

In further embodiments, the first and second health care information access rules are based on health care information categories and relationship status roles between the requesting source and the patient.

In still further embodiments, the first governmental administrative authority is a federal government administrative authority and the second governmental administrative authority is a state government administrative authority.

In some embodiments of the inventive concept, a computer program product comprises a non-transitory computer readable storage medium comprising computer readable program code embodied in the medium that is executable by a processor to perform operations comprising: defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority; receiving information associated with health care services provided to a patient; receiving a request to access a portion of the information from a requesting source; and determining whether to grant the request based on the portion of the information, the requesting source, and the first and second health care information access rules.

In other embodiments, the first and second health care information access rules are based on health care information categories and relationship status roles between the requesting source and the patient.

In still other embodiments, the first governmental administrative authority is a federal government administrative authority and the second governmental administrative authority is a state government administrative authority.

In further embodiments of the inventive concept, a method comprises receiving a health care information access rule; deconstructing, using a policy logic language, the health care information access rule into an access rule logic expression based on one or more variables and one or more predicates in the health care information access rule; receiving user input for a discretionary one of the one or more predicates indicating an accessibility for health care information protected by the health care information access rule; and automatically generating computer readable program code that logically implements the health care information access rule based on the access rule logic expression and the user input.

In still further embodiments, the policy logic language is written in eXtensible Access Control Markup Language (XACML).

In still further embodiments, the logic expression comprises a non-discretionary one or more predicates.

In still further embodiments, the non-discretionary one or more predicates is associated with a governmental authority.

In still further embodiments, the user input for the discretionary one or more predicates is based on a business policy not enforceable by a governmental authority.

In still further embodiments, the user input for the discretionary one or more predicates comprises a received patient preference for allowing access to the patient's health care information.

It is noted that aspects described with respect to one embodiment may be incorporated in different embodiments although not specifically described relative thereto. That is, all embodiments and/or features of any embodiments can be combined in any way and/or combination. Moreover, other methods, systems, articles of manufacture, and/or computer program products according to embodiments of the inventive concept will be or become apparent to one with skill in the art upon review of the following drawings and detailed description. It is intended that all such additional systems, methods, articles of manufacture, and/or computer program products be included within this description, be within the scope of the present inventive subject matter, and be protected by the accompanying claims. It is further intended that all embodiments disclosed herein can be implemented separately or combined in any way and/or combination.

### BRIEF DESCRIPTION OF THE DRAWINGS

Other features of embodiments will be more readily understood from the following detailed description of specific embodiments thereof when read in conjunction with the accompanying drawings, in which:

FIG. 1 is a block diagram of a system for providing access to a patient's health care information including a patient's delegated entities in accordance with some embodiments of the inventive concept;

FIG. 2 is a block diagram that illustrates a communication network including a system for providing access to a patient's health care information to the patient including the patient's delegated entities in accordance with some embodiments of the inventive concept;

FIG. 3 is a flowchart that illustrates operations for providing access to a patient's health care information to the patient including the patient's delegated entities in accordance with some embodiments of the inventive concept;

FIGS. 4 and 5 are user interfaces that illustrate a rule configuration platform for managing and creating non-discretionary and discretionary health care information access rules according to some embodiments of the inventive concept;

FIGS. 6-9 are flowcharts that illustrate further operations for providing access to a patient's health care information to the patient including the patient's delegated entities in accordance with some embodiments of the inventive concept;

FIG. 10 is a user interface that illustrates a rule configuration platform for managing and creating discretionary health care information access rules according to some embodiments of the inventive concept;

FIG. 11 is a data processing system that may be used to implement one or more servers in the health care information access system of FIG. 2 in accordance with some embodiments of the inventive concept; and

FIG. 12 is a block diagram that illustrates a software/hardware architecture for use in the health care information access system of FIG. 2 in accordance with some embodiments of the inventive concept.

### DETAILED DESCRIPTION

In the following detailed description, numerous specific details are set forth to provide a thorough understanding of embodiments of the present inventive concept. However, it will be understood by those skilled in the art that the present invention may be practiced without these specific details. In some instances, well-known methods, procedures, components, and circuits have not been described in detail so as not to obscure the present inventive concept. It is intended that all embodiments disclosed herein can be implemented separately or combined in any way and/or combination. Aspects described with respect to one embodiment may be incorporated in different embodiments although not specifically described relative thereto. That is, all embodiments and/or features of any embodiments can be combined in any way and/or combination.

As used herein, the term “provider” may mean any person or entity involved in providing health care services to a patient.

Some embodiments of the inventive concept stem from a realization that interoperability mandates in which payors and/or other entities are required to provide patients electronic access to their health care information/data carry with them the additional burden of ensuring that the health care information is handled properly and not disclosed to individuals that are not permitted to access the health care information. There may be a hierarchy of laws, regulations, and/or rules that govern the handling of access to patient health care information including, for example, laws, regulations, and/or rules associated with the federal government, state governments, and/or local (e.g., county, town, and/or municipality) governments. Embodiments of the inventive concept may provide a rule configuration platform by which non-discretionary rules can be developed and maintained based on laws, regulations, and/or rules promulgated by the hierarchy of governing authorities. The rule configuration platform may provide a user interface that allows an administrative user to select a health care information access restriction associated with a health care information category and a patient relationship status role. In accordance with various embodiments of the inventive concept, the patient relationship status roles may include familial relationship entities, such as the patient, the patient's spouse, and/or the patient's child, may include entities with an agency relationship with the patient, such as a patient's physician or caretaker, and/or may include third party entities, such as pharmacies, health care applications or websites. Because the different laws, regulations, and/or rules governing the handling and communication of health care information/data may be governed by a hierarchy of laws, the rule configuration platform may provide a way in which the relationships between those different governing authorities can be managed. For example, the authority that has priority over all others, i.e., the authority at the top of the hierarchy, may serve as a baseline rule set. Those governing authorities lower on the hierarchy, i.e., with less authority, may provide rule deviations from the baseline rule set that do not conflict with the baseline rule set. Thus, for example, various state governments may provide rules that are more restrictive than federal government rules or may provide permissive rules in areas in which the federal government has not defined a rule. Embodiments of the inventive concept may further provide a computer readable program code development platform that may reduce compliance risks in controlling access to health care information. These risks may be associated with multiple levels including legal risks of complying with non-discretionary rules, standard risks, including risks of complying with discretionary rules corresponding to business standards and/or patient preferences, and procedural risks including risks in developing computer readable program code. This may reduce errors in generating code for implementing and updating rules as the restrictions change over time and as the restrictions can vary between different entities in the hierarchy, e.g., from federal to state, from state to state, etc. These health care information access rules may be used when a party requests access to health care information for a patient in determining whether to grant the request.

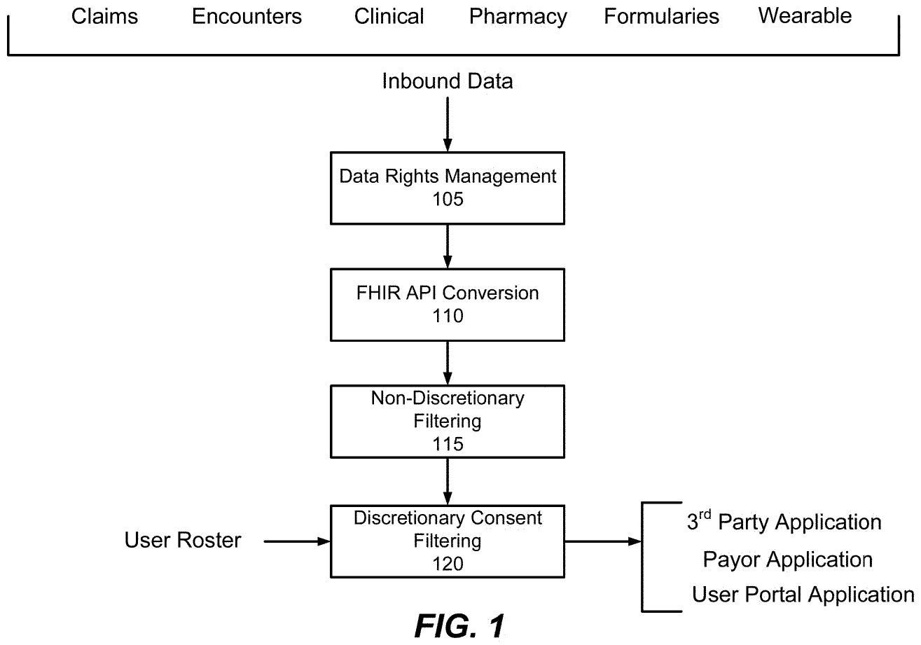

FIG. 1 is a block diagram of a system for providing access to a patient's health care information including a patient's delegated entities in accordance with some embodiments of the inventive concept. As shown in FIG. 1, the system may receive information associated with health care services provided to a patient. The information may come from a variety of source and may include, but is not limited to, payor claim information, encounter information, clinical information, pharmacy information, formulary information, and/or wearable information. The payor claim information may include claim information associated with a current payor (e.g., insurance company) that pays claims for the patient and one or more former payors that paid claims for the patient in the past. The wearable information may be generated by devices, such as watches, wrist worn fitness trackers, rings, body worn sensors, and the like. The inbound health care information/data may then be processed at block 105 to ensure compliance with any data rights management agreements that may be governing the use of and/or access to the received health care information. In accordance with some embodiments of the inventive concept, the digital rights management agreements may be enforced using the apparatus and method described in U.S. patent application Ser. No. 16/353,507 entitled “Apparatus and Method Configured to Facilitate the Selective Search of a Database,” filed Mar. 14, 2019 the disclosure of which is hereby incorporated herein by reference.

The received health care information may be further processed at block 110 by way of conversion into a format compatible with the FHIR protocol. The FHIR protocol is a standard that describes the data formats, elements/resources, and an application programming interface (API) for exchanging electronic health records and information. Use of a standardized protocol may assist third parties in developing software to process the health care information in response to access requests.

Embodiments of the inventive concept may provide a system for providing access to patient health care information that includes both a non-discretionary patient information access filtering at block 115 and discretionary patient information access or consent filtering at block 120. The non-discretionary patient information access filtering may be used to ensure compliance with mandatory health information access laws, regulations, and/or rules issued by, for example, governmental authorities. The non-discretionary patient information access filtering may provide a hierarchical filtering structure in which the health information access rules may be associated with different administrative authorities having different precedence or priority levels with respect to each other. For example, the different administrative authorities may be different governmental authorities, such as the federal government, state governments, local/municipality governments, etc. The discretionary patient information access or consent filtering may be used by a patient to configure a select group of entities (e.g., family members, third party applications, payor applications, user portal application, etc.) that are allowed access to the patient's health care information including the specific information categories or types of health care information that the entities are allowed to access. The discretionary access rules configured by the patient are subservient to the non-discretionary rules mandated by some administrative authority, such as one or more government entities. Thus, the effect of the non-discretionary filtering of block 115 and the discretionary filtering of block 120 is to create a hierarchy of rules that can ensure compliance with interoperability mandates to allow patients to electronically access their health care information, while ensuring that the access does not violate any laws governing the handling and/or communication of health care information, but providing the patient with flexibility to customize what information is accessible by particular delegated entities. It will be understood that in accordance with various embodiments of the inventive concept, a patient's delegated entities may be the result of a selection by the patient or the operation of law. For example, by operation of law a child's health care information may be accessible by a parent or guardian irrespective of whether the child grants the parent or guardian permission to access the health care information.

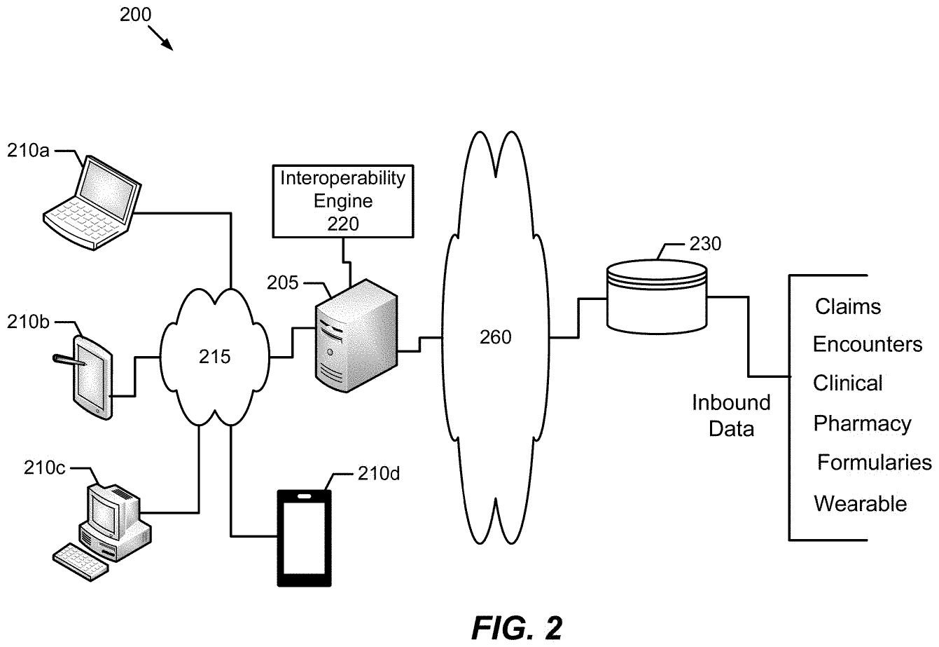

Referring to FIG. 2, a communication network 200 including a system for providing access to a patient's health care information to the patient including the patient's delegated entities, in accordance with some embodiments of the inventive concept, comprises an interoperability server 205 that is coupled to devices 210a, 210b, 210c, and 210d via network 215. The interoperability server 205 may be configured with an interoperability engine module 220 to provide access to a patient's health care information to the patient including the patient's delegated entities in response to requests therefrom. The interoperability engine module 220 may be configured with one or more sub-modules that are configured to implement the functionality of the data rights management block 105, the FHIR API conversion block 110, the non-discretionary filtering block 115, and the discretionary consent filtering block 120 of FIG. 1.

The interoperability server 205 is configured to receive information associated with health care services provided to a patient. As described above, the information may include, but is not limited to, payor claim information, encounter information, clinical information, pharmacy information, formulary information, and/or wearable information. This information may be stored in a database 230 located, for example, in the cloud to be accessed by the interoperability server 205 over the network 260. The network 260 couples the health care patient information sources and the database 230 containing the patient health care information/data to the interoperability server 205. The network 260 may be a global network, such as the Internet or other publicly accessible network. Various elements of the network 260 may be interconnected by a wide area network, a local area network, an Intranet, and/or other private network, which may not be accessible by the general public. Thus, the communication network 260 may represent a combination of public and private networks or a virtual private network (VPN). The network 260 may be a wireless network, a wireline network, or may be a combination of both wireless and wireline networks.

The network 215 communicatively couples the devices 210a, 210b, 210c, and 210d to the interoperability server 205. The network 215 may comprise one or more local or wireless networks and/or one or more wide area or global networks, such as the Internet to facilitate communication between the interoperability server 205 and the devices 210a, 210b, 210c, and 210d. The devices 210a, 210b, 210c, and 210d may be used by a patient, a patient's agent, and/or delegates of the patient to submit requests to the interoperability server 205 to access the patient's health care information and to receive the patient's health care information in response to these requests.

Although FIG. 2 illustrates an example communication network an interoperability system for providing access to a patient's health care information to the patient including the patient's delegated entities, it will be understood that embodiments of the inventive subject matter are not limited to such configurations, but are intended to encompass any configuration capable of carrying out the operations described herein.

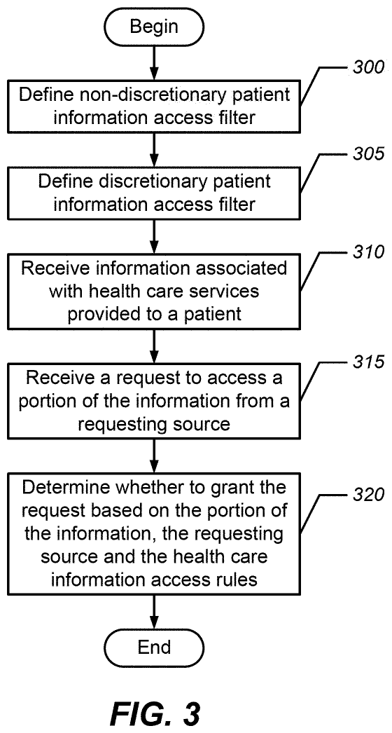

FIG. 3 is a flowchart that illustrates operations for providing access to a patient's health care information to the patient including the patient's delegated entities in accordance with some embodiments of the inventive concept. Operations begin at block 300 where a non-discretionary patient information access filter is defined. At block 305 a discretionary patient information access filter is defined. Information associated with health care services provided to a patient is received at block 310. This information may include, but is not limited to, payor claim information, encounter information, clinical information, pharmacy information, formulary information, and/or wearable information. A request is received at block 315 to access a portion of the patient's health care information from a requesting source. The requesting source may be any number of entities including the patient, a person with a familial relationship with the patient, an agent of the patient, a service provider for the patient, e.g., a physician, therapist, etc., a business entity, such as a pharmacy or insurance claim payor, and/or any number of websites or applications, such as an insurance payor website, a fitness application, a hospital or clinic website, or the like. The requesting source may also be a hostile entity attempting to access the patient's health care information with malicious intent. A determination is made at block 320 whether to grant the request based on the portion of the information requested, the requesting source (e.g., an identity of the requesting source), and the health care information access rules based on the non-discretionary patient information access filter and the discretionary patient information access filter.

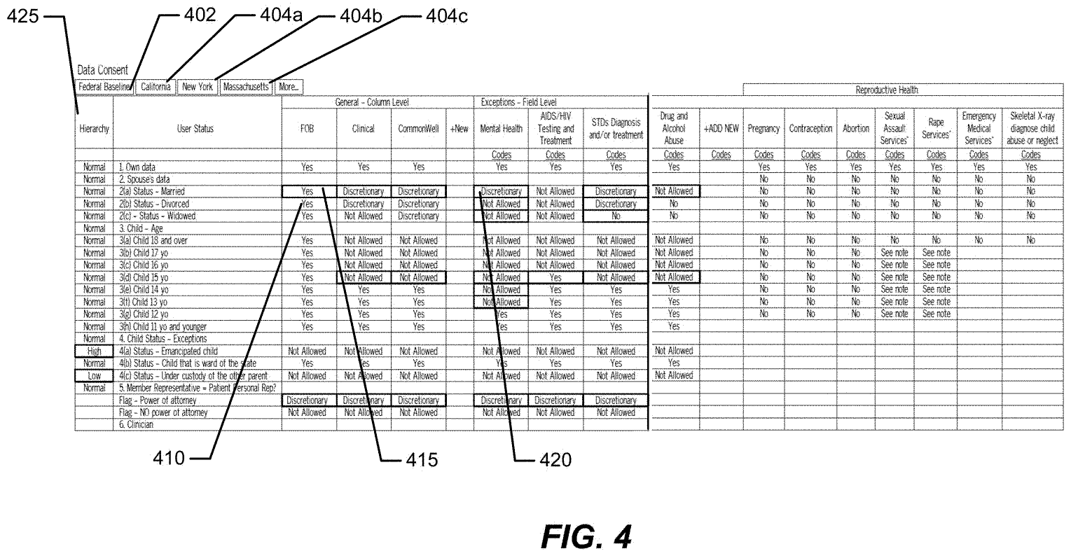

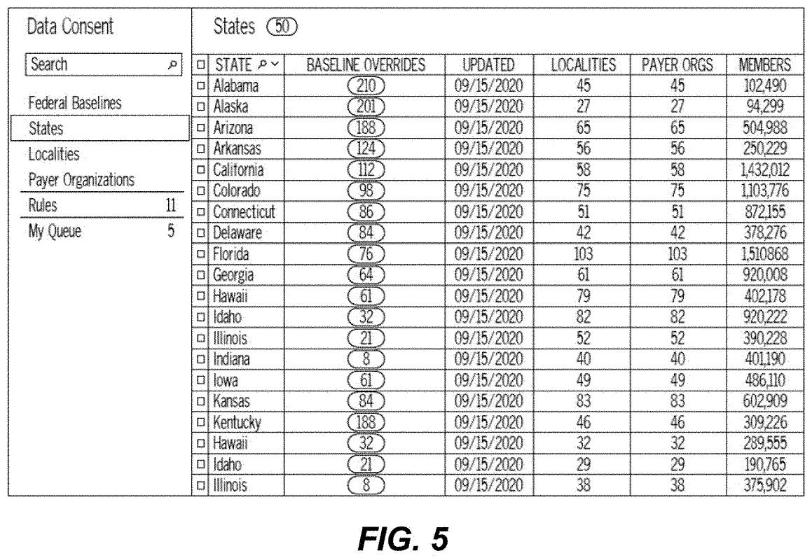

FIGS. 4 and 5 are user interfaces that illustrate a rule configuration platform for managing and creating non-discretionary and discretionary health care information access rules according to some embodiments of the inventive concept. Referring to FIG. 4, the rule configuration platform may provide a user interface in the form of a spreadsheet. In the example shown, the columns may represent health care information categories, such as Explanation of Benefits (EOB) information (claims information), clinical information, and various clinical health care information categories ranging from mental health to abortion. The rows may represent a particular relationship status role between the patient and a requesting source. For example, the role may be “self” or “own data” if the requesting source is the patient. As shown in FIG. 4, numerous rows are defined for the requesting source having a familial relationship with the patient and the particular status or age of the patient.

As illustrated by the tabs, the rule configuration platform may support a hierarchy of non-discretionary rules. The rules emanating from the entity in the hierarchy with the highest priority or greatest authority may serve as a baseline 402. In the example shown, the baseline rules may correspond to laws, regulations and/or rules issued by the federal government. Other entities lower in the hierarchy may also provide rules that may coexist, but may not conflict with the rules associated with the entities higher up in the hierarchy, i.e., having greater priority. In the example shown, additional rules may be supported that are associated with various individual states as represented by tabs 404a, 404b, and 404c. As shown in FIG. 4, health care information for a patient in California is protected by all federal rules. An example of which is under federal law a person is allowed to access his or her divorced spouse's EOB health care information as highlighted in cell 410. With respect to a married couple, California explicitly allows access to a spouse's EOB health care information and this law or regulation does not conflict with any federal law or regulation at highlighted in cell 415. The entry associated with cell 415 may be considered a baseline override of the federal rules that are applicable to every state. FIG. 5 illustrates a user interface showing the number of baseline overrides that supplement the federal rules for a variety of different states. Thus, the state rules may supplement the federal rules to be more restrictive in granting access to information than the federal rules or to fill the gap where no federal rule is on point, but the state rules may not conflict with the federal rules. Returning to FIG. 4, column 425 provides a hierarchy field, which may be used to resolve conflicts between state rules that conflict with one another to determine which rule takes precedence. In the example shown in FIG. 4, a rule may be assigned a precedence of high, normal, or low. The rule configuration platform of FIG. 4 may provide a mechanism by which non-discretionary rules associated with a hierarchy of authorities may be managed to control access to patient's health care information while supporting interoperability mandates. The rule configuration platform of FIG. 4 may also be used to track the status of discretionary rules, which may be based on business policies or patient preferences, in areas where the non-discretionary rules permit flexibility or are silent. For example, the entry associated with cell 420 indicates that a payor has the flexibility to implement its own policy with respect to the release of that information including, for example, providing the patient with the ability to choose whether to access the health care information and/or allow other entities to access the health care information.

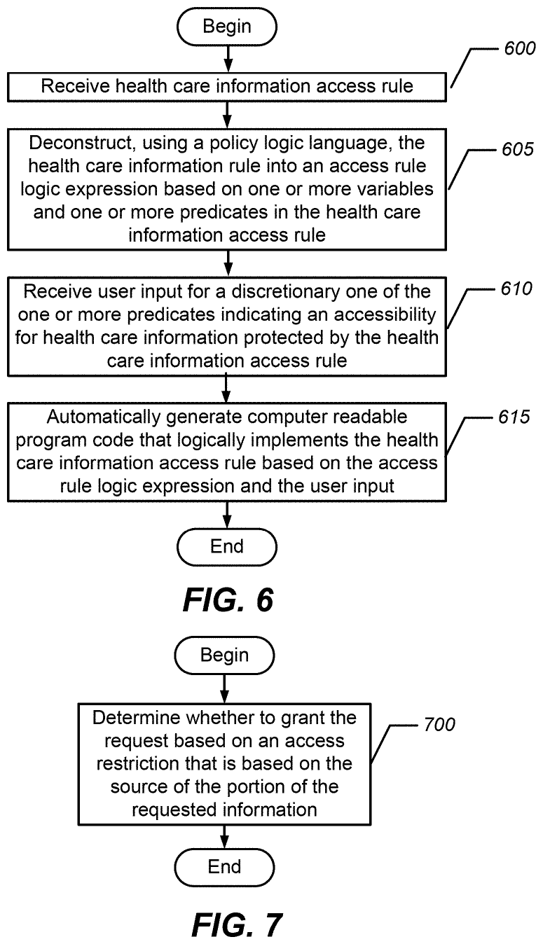

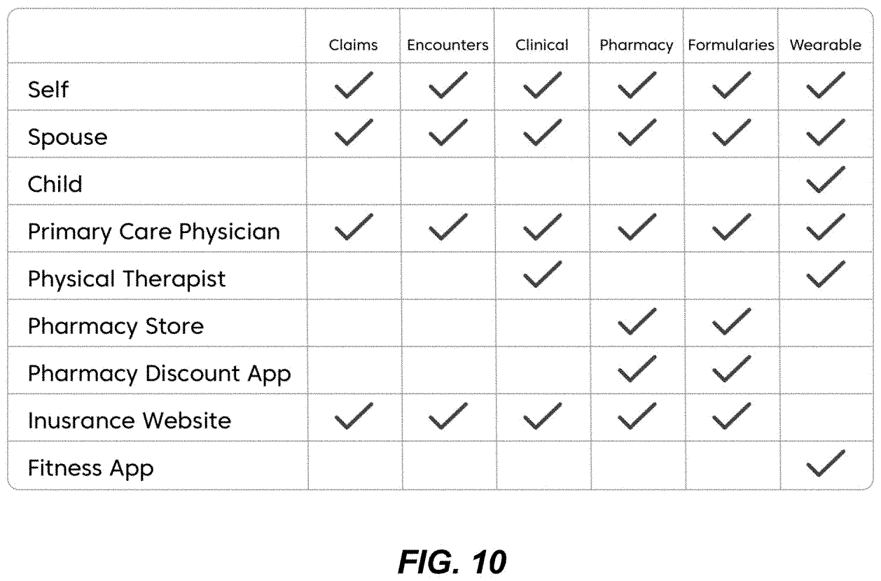

FIGS. 6-9 are flowcharts that illustrate further operations for providing access to a patient's health care information to the patient including the patient's delegated entities in accordance with some embodiments of the inventive concept. Referring now to FIGS. 6 and 4, some embodiments of the inventive concept may provide a system for the automatic generation of computer readable program code ensuring compliance with risks in controlling access to health care information at multiple levels including legal risks of complying with non-discretionary rules and regulations set forth by governmental entities, standards bodies, the like, standard risks including risks of complying with discretionary rules corresponding to business standards and/or patient preferences, and procedural risks including risks in developing computer readable program code for implementing systems that control access to the health care information. Embodiments of the inventive concept may provide a computer readable code development platform that may reduce the aforementioned risks in providing access to health care information requested by a patient and those entities that a patient chooses to allow access to the patient's health care information. Operations begin at block 600 where one or more health care information access rules are received. Each of these rules may be a rule from a governmental entity, such as the federal, state, or local governmental entity, a standards body, or the like. Each rule may be deconstructed or parsed using a policy logic language in which the variables and predicates within the rule are identified and the requirements of the rule are expressed via a logic expression at block 605. The rules may be deconstructed or parsed using a variety of techniques including, for example, Artificial Intelligence (AI) techniques, such as Natural Language Processing (NLP), which can be trained to identify the variables and predicates in the rules. In some embodiments, the policy logic language may be implemented using eXtensible Access Control Markup Language (XACML). It will be understood, however, that other types of policy description languages may be used in accordance with various embodiments of the inventive concept. The predicate(s) in the logic expression may include one or more non-discretionary predicates and/or one or more discretionary predicates. The discretionary predicates may be derived explicitly from the rule's language or may be derived from an area in which the rule does not speak leaving other entities the option of developing their own rules or requirements in that area or space. At block 610, user input may be received for one or more discretionary predicates indicating an accessibility for health care information protected by the corresponding health care information access rule. This is illustrated, for example, with respect to the rule configuration platform of FIG. 4 in which input can be provided via an administrator to modify a discretionary predicate to carry out a business policy with respect to granting access to health care information (e.g., cell 420 of FIG. 4) that is not restricted by mandatory rules stemming from, for example, a governmental authority. These business policies may not be enforceable by a governmental authority and may be implemented at the discretion of the payor, for example. The payor may also choose to allow the patient to select which entities are allowed to receive the patient's health care information as described below with respect to FIG. 10. This input from the patient may also be used to modify a discretionary predicate to implement the patient's preference with respect to access to the patient's health care information. Computer readable code that logically implements the health care information access rule(s) may be generate at block 615 based on the access rule logic expression and the user input. Through use of the policy logic language, which can be verified to accurately represent the logical requirements, including both discretionary and non-discretionary aspects of a health care information access rule (e.g., law, regulation, rule, etc.), and limiting user input to choosing accessibility options for discretionary predicates in the rules, may reduce errors in generating computer readable program code used to implement policies regarding controlling access to healthcare information. Thus, the overall governance of complying with governmental, business, and patient requirements can be evaluated and shown to be correct, which can be essential when handling sensitive information, such as patient healthcare data. Embodiments of the inventive concept may also enforce the hierarchy between the various non-discretionary rule authorities to ensure that a rule does not conflict with another rule that has higher precedence, such as a baseline rule corresponding to a federal rule in the example of FIG. 4.

As described above, the inbound health care information/data may be processed to ensure compliance with any data rights management agreements that may be governing the use of and/or access to the received health care information. Referring now to FIG. 7, at block 700, a determination whether to grant a request for patient health care information is based on an access restriction that is based on the source of the portion of the requested information. For example, a source of certain types of health care information may have contractual agreements on what entities may access the health care information and other restrictions on how the health care information may be used. Embodiments of the inventive concept may be configured to support the digital rights management restrictions that may be associated with various types of health care information received for a patient.

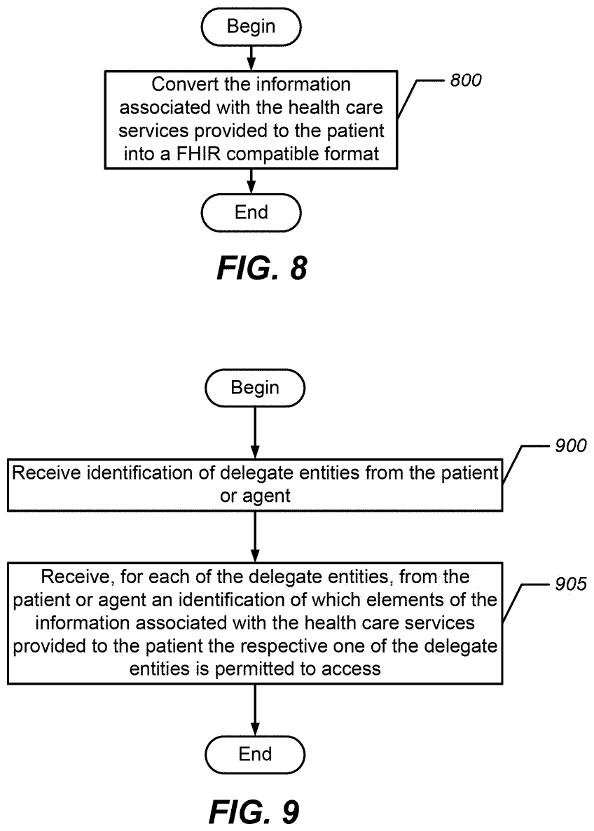

Referring now to FIG. 8, received health care information may be processed at block 800 by way of conversion into a format compatible with the FHIR protocol. Conversion of the health care information into a standardized protocol, such as the FHIR protocol, may facilitate the development of applications to process the health care information to provide patients and their delegates electronic access to the patients' health care information as part of supporting interoperability.

As described above, the discretionary filtering capability may allow a patient the flexibility to customize what entities are able to access the patient's health care information while the non-discretionary filtering capability ensures compliance with all mandatory laws, regulations, and/or rules. FIGS. 9 and 10 illustrate further operations of the discretionary filtering capability that may allow a patient to tailor what information is accessible to which entities subject to the non-discretionary rules. Operations begin at block 900 where the patient or patient's agent identifies one or more delegate entities. This is illustrated in FIG. 10 where each row corresponds to a specific delegate entity including the patient, the patient's spouse, the patient's child, a primary care physician, a physical therapist, a pharmacy store, a pharmacy discount application, an insurance website, and a fitness application. These delegate entities are examples and other entities may be defined in accordance with various embodiments of the inventive concept. At block 905, the patient or the patient's agent may identify which elements of information associated with the health care services provided to the patient may be provided to the different delegate entities. Referring to FIG. 10, in the example shown the health care information associated with services provided to the patient includes claims information, encounter information, clinical information, pharmacy information, formulary information, and wearable device information. The patient has indicated that all categories of information are accessible to the patient's spouse and the patient's primary care physician. The patient is only willing to share information from the patient's wearable device with the patient's child and with the patient's fitness application. The physical therapist is given permission to access the clinical information along with the wearable device information. The pharmacy store and the pharmacy discount application are permitted to access both pharmacy and formulary information. The insurance website is permitted to access all information except for the wearable device information. While broad categories of health care information are used in the example of FIG. 10, embodiments of the inventive concept are not limited to any particular granularity of patient health care information. In some embodiments of the inventive concept, the patient may create a policy for health care information access that applies a defined access rule for one entity to another entity. For example, a user may define a certain level of health care information access for the patient's current health insurance provider (first payor). If the patient changes health insurance companies in the future, this health care information access policy that was applied to the patient's first payor may automatically be applied to the patient's new insurance company (second payor). Embodiments of the inventive concept may also provide a mechanism for sharing health care information among family members using a single interface. For example, a family may have various members that use different insurance companies or payors and see different health care practitioners. The family members could make each other delegates allowing parents, for example, to access the health care information for all family members through one interface across multiple payors and health care service providers.

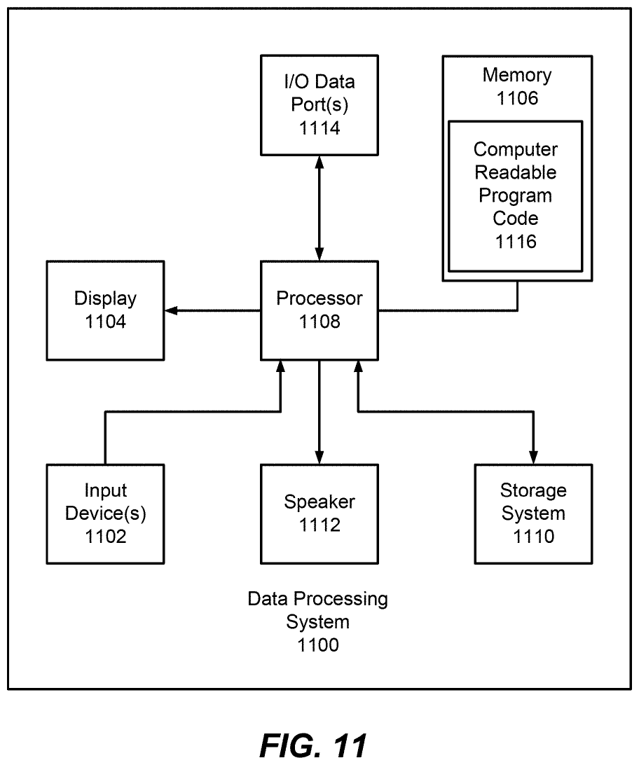

Referring now to FIG. 11, a data processing system 1100 that may be used to implement the interoperability server 205 of FIG. 2, in accordance with some embodiments of the inventive concept, comprises input device(s) 1102, such as a keyboard or keypad, a display 1104, and a memory 1106 that communicate with a processor 1108. The data processing system 1100 may further include a storage system 1110, a speaker 1112, and an input/output (I/O) data port(s) 1114 that also communicate with the processor 1108. The processor 1108 may be, for example, a commercially available or custom microprocessor. The storage system 1110 may include removable and/or fixed media, such as floppy disks, ZIP drives, hard disks, or the like, as well as virtual storage, such as a RAMDISK. The I/O data port(s) 1114 may be used to transfer information between the data processing system 1100 and another computer system or a network (e.g., the Internet). These components may be conventional components, such as those used in many conventional computing devices, and their functionality, with respect to conventional operations, is generally known to those skilled in the art. The memory 1106 may be configured with computer readable program code 1116 to provide access to a patient's health care information to the patient including the patient's delegated entities in accordance with some embodiments of the inventive concept.

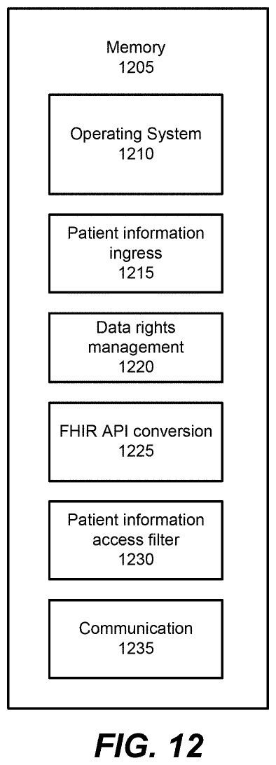

FIG. 12 illustrates a memory 1205 that may be used in embodiments of data processing systems, such as the interoperability server 205 of FIG. 2 and the data processing system 1100 of FIG. 11, respectively, to provide access to a patient's health care information to the patient including the patient's delegated entities in accordance with some embodiments of the inventive concept. The memory 1205 is representative of the one or more memory devices containing the software and data used for facilitating operations of the interoperability server 205 as described herein. The memory 1205 may include, but is not limited to, the following types of devices: cache, ROM, PROM, EPROM, EEPROM, flash, SRAM, and DRAM. As shown in FIG. 12, the memory 1205 may contain six or more categories of software and/or data: an operating system 1210, a patient information ingress module 1215, a data rights management module 1220, an FHIR API conversion module 1225, a patient information access filter 1230, and a communication module 1235. In particular, the operating system 1210 may manage the data processing system's software and/or hardware resources and may coordinate execution of programs by the processor. The patient information ingress module 1215, the data rights management module 1220, the FHIR API conversion module 1225, the patient information access filter 1230, and the communication module 1235 may be configured to perform one or more operations described above with respect to the interoperability server 205 and the patient health care information access system of FIG. 1. In some embodiments, the patient information ingress module 1215 may be configured to perform one or more of the operations described above with respect to block 310 of FIG. 3, the data rights management module 1220 may be configured to perform one or more of the operations described above with respect to block 105 of FIG. 1 and block 700 of FIG. 7, the FHIR API conversion module 1225 may be configured to perform one or more of the operations described above with respect to block 110 of FIG. 1 and block 800 of FIG. 8. The patient information access filter 1230 may be configured to perform one or more of the operations described above with respect to blocks 115 and 120 of FIG. 1, blocks 300 and 320 of FIG. 3, blocks 600, 605, and 610 of FIG. 6, and blocks 900 and 905 of FIG. 9. The communication module 1235 may be configured to perform one or more operations described above with respect to block 310 of FIG. 3, block 605 of FIG. 6, and blocks 900 and 905 of FIG. 9.

Although FIGS. 11-12 illustrate hardware/software architectures that may be used in data processing systems, such as the interoperability server 205 of FIG. 2 and the data processing system 1100 of FIG. 11, respectively, in accordance with some embodiments of the inventive concept, it will be understood that the present invention is not limited to such a configuration but is intended to encompass any configuration capable of carrying out operations described herein.

Computer program code for carrying out operations of data processing systems discussed above with respect to FIGS. 1-12 may be written in a high-level programming language, such as Python, Java, C, and/or C++, for development convenience. In addition, computer program code for carrying out operations of the present invention may also be written in other programming languages, such as, but not limited to, interpreted languages. Some modules or routines may be written in assembly language or even micro-code to enhance performance and/or memory usage. It will be further appreciated that the functionality of any or all of the program modules may also be implemented using discrete hardware components, one or more application specific integrated circuits (ASICs), or a programmed digital signal processor or microcontroller.

Moreover, the functionality of the interoperability server 205 of FIG. 2 and the data processing system 1100 of FIG. 11 may each be implemented as a single processor system, a multi-processor system, a multi-core processor system, or even a network of stand-alone computer systems, in accordance with various embodiments of the inventive concept. Each of these processor/computer systems may be referred to as a “processor” or “data processing system.”

The data processing apparatus described herein with respect to FIGS. 1-12 may be used to provide access to a patient's health care information to the patient including the patient's delegated entities according to some embodiments of the inventive concept described herein. These apparatus may be embodied as one or more enterprise, application, personal, pervasive and/or embedded computer systems and/or apparatus that are operable to receive, transmit, process and store data using any suitable combination of software, firmware and/or hardware and that may be standalone or interconnected by any public and/or private, real and/or virtual, wired and/or wireless network including all or a portion of the global communication network known as the Internet, and may include various types of tangible, non-transitory computer readable media. In particular, the memory 1205 when coupled to a processor includes computer readable program code that, when executed by the processor, causes the processor to perform operations including one or more of the operations described herein with respect to FIGS. 1-10.

Some embodiments of the inventive concept may provide a system that supporter interoperability to provide patients and their delegates access to their health care information while ensuring through use of non-discretionary filtering that the health care information is handled in a secure manner that does not violate and laws, regulations, and/or rules governing the handling and/or the communication of the health care information. The non-discretionary rules may be managed through a rule configuration platform that provides for increased accuracy in implementing the rules through automated code generation via the administrative user interface. Moreover, embodiments of the inventive concept may provide discretionary rules that may be configured by a patient to define various delegates and to specify what types of health care information from which information sources that delegates can access. In this way, a patient can control access to what information sources the patient wishes to see including clinical and claims information from current and former providers and payors, for example. The patient may also manage the health care information for the patient's entire family through use of delegates that allow family members to view each other's health care information even through the family members may use different payors and/or see different providers.

### FURTHER DEFINITIONS AND EMBODIMENTS

In the above-description of various embodiments of the present inventive concept, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this inventive concept belongs. It will be further understood that terms, such as those defined in commonly used dictionaries, should be interpreted as having a meaning that is consistent with their meaning in the context of this specification and the relevant art and will not be interpreted in an idealized or overly formal sense expressly so defined herein.

The flowchart and block diagrams in the figures illustrate the architecture, functionality, and operation of possible implementations of systems, methods, and computer program products according to various aspects of the present inventive concept. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular aspects only and is not intended to be limiting of the inventive concept. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Like reference numbers signify like elements throughout the description of the figures.

In the above-description of various embodiments of the present inventive concept, aspects of the present inventive concept may be illustrated and described herein in any of a number of patentable classes or contexts including any new and useful process, machine, manufacture, or composition of matter, or any new and useful improvement thereof. Accordingly, aspects of the present inventive concept may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present inventive concept may take the form of a computer program product comprising one or more computer readable media having computer readable program code embodied thereon.

Any combination of one or more computer readable media may be used. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

The description of the present inventive concept has been presented for purposes of illustration and description, but is not intended to be exhaustive or limited to the inventive concept in the form disclosed. Many modifications and variations will be apparent to those of ordinary skill in the art without departing from the scope and spirit of the inventive concept. The aspects of the inventive concept herein were chosen and described to best explain the principles of the inventive concept and the practical application, and to enable others of ordinary skill in the art to understand the inventive concept with various modifications as are suited to the particular use contemplated.

What is claimed is:

1. A method, comprising:
performing on a processor:
defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority;
receiving information associated with health care services provided to a patient;
receiving a request to access a portion of the information from a requesting source; and
determining whether to grant the request based on the portion of the information, the requesting source, and the first and second health care information access rules.

2. The method of claim 1, wherein the first and second health care information access rules are based on health care information categories and relationship status roles between the requesting source and the patient.

3. The method of claim 2, wherein the health care information categories comprise a claim information category, an encounter information category, a clinical information category, pharmacy information category, a formulary information category, and a wearable information category.

4. The method of claim 3, wherein the claim information category comprises claim information associated with a current payor for the patient and a former payor for the patient.

5. The method of claim 3, wherein the clinical information category comprises a plurality of clinical conditions.

6. The method of claim 5, wherein each of the plurality of clinical conditions is associated with a plurality of clinical sub-conditions identifiable by a plurality of clinical codes, respectively.

7. The method of claim 6, wherein each of the second health care information access rules has a hierarchy rating associated therewith, the hierarchy rating specifying a precedence for resolving conflicts between ones of the second health care information access rules.

8. The method of claim 1, wherein the first governmental administrative authority is a federal government administrative authority and the second governmental administrative authority is a state government administrative authority.

9. A system, comprising:
a processor; and
a memory coupled to the processor and comprising computer readable program code embodied in the memory that is executable by the processor to perform operations comprising:
defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority;
receiving information associated with health care services provided to a patient;
receiving a request to access a portion of the information from a requesting source; and
determining whether to grant the request based on the portion of the information, the requesting source, and the first and second health care information access rules.

10. The system of claim 9, wherein the first and second health care information access rules are based on health care information categories and relationship status roles between the requesting source and the patient.

11. The system of claim 9, wherein the first governmental administrative authority is a federal government administrative authority and the second governmental administrative authority is a state government administrative authority.

12. A computer program product, comprising:
a non-transitory computer readable storage medium comprising computer readable program code embodied in the medium that is executable by a processor to perform operations comprising:
defining a non-discretionary patient information access filter, the non-discretionary patient information access filter comprising first health care information access rules associated with a first governmental administrative authority and second health care information access rules associated with a second governmental administrative authority, the first governmental administrative authority having priority over the second governmental administrative authority;
receiving information associated with health care services provided to a patient;
receiving a request to access a portion of the information from a requesting source; and
determining whether to grant the request based on the portion of the information, the requesting source, and the first and second health care information access rules.

13. The computer program product of claim 12, wherein the first and second health care information access rules are based on health care information categories and relationship status roles between the requesting source and the patient.

14. The computer program product of claim 12, wherein the first governmental administrative authority is a federal government administrative authority and the second governmental administrative authority is a state government administrative authority.

15. A method, comprising:
receiving a health care information access rule;
deconstructing, using a policy logic language, the health care information access rule into an access rule logic expression based on one or more variables and one or more predicates in the health care information access rule;
receiving user input for a discretionary one of the one or more predicates indicating an accessibility for health care information protected by the health care information access rule; and
automatically generating computer readable program code that logically implements the health care information access rule based on the access rule logic expression and the user input.

16. The method of claim 15, wherein the policy logic language is written in eXtensible Access Control Markup Language (XACML).

17. The method of claim 15, wherein the logic expression comprises a non-discretionary one or more predicates.

18. The method of claim 17, wherein the non-discretionary one or more predicates is associated with a governmental authority.

19. The method of claim 15, wherein the user input for the discretionary one or more predicates is based on a business policy not enforceable by a governmental authority.

20. The method of claim 15, wherein the user input for the discretionary one or more predicates comprises a received patient preference for allowing access to the patient's health care information.
